(19) United States
(12) Patent Application Publication
(10) Pub. No.: US 2018/0224416 A1
(43) Pub. Date: Aug. 9, 2018

(54) GAS SENSOR AND METHOD OF MANUFACTURING THE SAME

(71) Applicant: Winbond Electronics Corp., Taichung City (TW)

(72) Inventors: Ming-Chih Tsai, Taichung City (TW); Yu-Hsuan Ho, Taichung City (TW)

(73) Assignee: Winbond Electronics Corp., Taichung City (TW)

(21) Appl. No.: 15/695,018

(22) Filed: Sep. 5, 2017

(30) Foreign Application Priority Data
Feb. 3, 2017 (CN) 201710063478.2

(51) Int. Cl. G01N 33/00; G01N 27/327; B01D 71/16; B01D 71/28; B01D 71/02; B01D 69/02; B82Y 15/00

(52) U.S. Cl. CPC G01N 33/0075; G01N 27/3278; B01D 71/16; B82Y 15/00; B01D 71/021; B01D 71/025; B01D 69/02; B01D 71/28

(57) ABSTRACT

A gas sensor including a substrate, an output layer, a sensing layer, and a nanoporous polymer film is provided. The output layer is disposed on the substrate. The sensing layer is disposed on the output layer. The nanoporous polymer film is disposed on the sensing layer.

### CROSS-REFERENCE TO RELATED APPLICATION

This application claims the priority benefit of China application serial no. 201710063478.2, filed on Feb. 3, 2017. The entirety of the above-mentioned patent application is hereby incorporated by reference herein and made a part of this specification.

### BACKGROUND OF THE INVENTION

### Field of the Invention

The invention relates to a gas sensor and a method of manufacturing the same, and more particularly, to a gas sensor having a nanoporous polymer film and a method of manufacturing the same.

### Description of Related Art

In the known gas sensor, a material having microchannels is disposed on the sensing layer such that gas molecules having a size smaller than the microchannels pass through the microchannels to be in contact with the sensing layer. On the other hand, gas molecules having a size greater than the microchannels cannot pass through the microchannels and therefore cannot be in contact with the sensing layer.

However, a known material having microchannels is anodized aluminum oxide. Since anodized aluminum oxide is a metal oxide material, the anodized aluminum oxide and the sensing layer do not fit, and therefore the issue of insufficient durability of the gas sensor readily occurs.

### SUMMARY OF THE INVENTION

The invention provides a gas sensor and a method of manufacturing the same having very good durability and performance.

The invention provides a gas sensor including a substrate, an output layer, a sensing layer, and a nanoporous polymer film. The output layer is disposed on the substrate. The sensing layer is disposed on the output layer. The nanoporous polymer film is disposed on the sensing layer.

The invention also provides a method of manufacturing a gas sensor including: forming an output layer on a substrate; forming a sensing layer on the output layer; and forming a nanoporous polymer film on the sensing layer.

Based on the above, the nanoporous polymer film of the invention has small holes selectively allowing smaller molecules to pass through and blocking larger molecules. Moreover, the nanoporous polymer film is located on the sensing layer of the gas sensor and can provide better protection to the sensing layer. Therefore, the gas sensor of the invention has very good durability and performance.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic diagram of a gas sensor of an embodiment of the invention.

FIG. 2 is a cross-sectional schematic diagram of a gas sensor of an embodiment of the invention.

FIG. 3 is a flow chart of a method of manufacturing a gas sensor of an embodiment of the invention.

### DESCRIPTION OF THE EMBODIMENTS

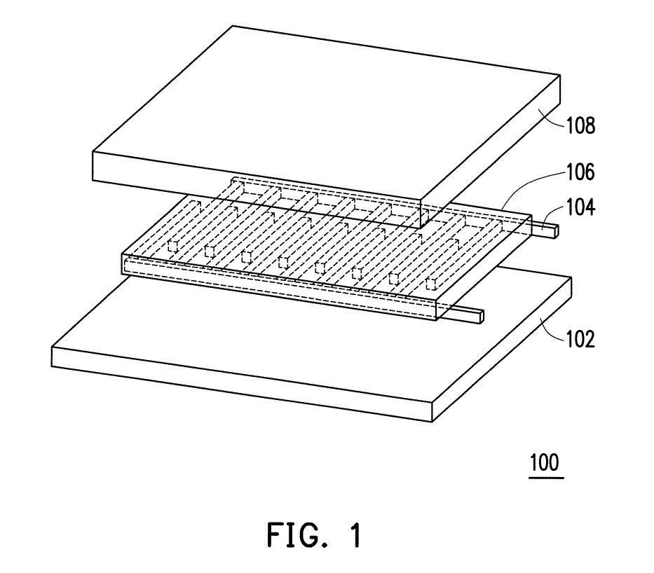

FIG. 1 is a schematic diagram of a gas sensor of an embodiment of the invention. Referring to FIG. 1, a gas sensor 100 includes: a substrate 102 and an output layer 104, a sensing layer 106, and a nanoporous polymer film 108 disposed from the bottom up. After the gas molecules pass through the nanoporous polymer film 108, the gas molecules interact with the sensing layer 106 below to change the resistance of the sensing layer 106. The output layer 104 receives a signal produced by the change in the physical properties (such as resistance, capacitance, or impedance) of the sensing layer 106 and can learn the resistance change of the sensing layer 106 according to the detected signal so as to learn the type, composition, or amount of the detected gas molecules.

The surface of the substrate 102 can be a flat surface, a non-planar surface, or a combination thereof. The flat surface can be a smooth surface or a rough surface. The non-planar surface can be a convex surface, a concave surface, a double concave surface, or a double convex surface. In an embodiment in which the substrate 102 is a non-planar surface, the gas sensor 100 can be disposed at a non-planar surface location or body, and therefore has a broader application scope. The substrate 102 can be a flexible material or a rigid material. The material of the substrate 102 is, for instance, glass, poly(ethylene terephthalate) (PET), polyethylene naphthalate (PEN), polyimide (PI), polyvinyl chloride (PVC), polypropylene (PP), cycloolefin polymer (COP), polyethylene (PE), or a combination thereof.

The output layer 104 is disposed on the substrate 102. The output layer 104 can measure the physical properties (such as resistance, capacitance, or impedance) of the sensing layer 106. In some embodiments, the output layer 104 can include an electrode, a switch (such as a thin-film transistor, a bipolar junction transistor (BJT), or a diode), or a combination thereof. In an embodiment in which the output layer 104 can be an electrode, the electrode can receive a signal and send the signal to a detection device.

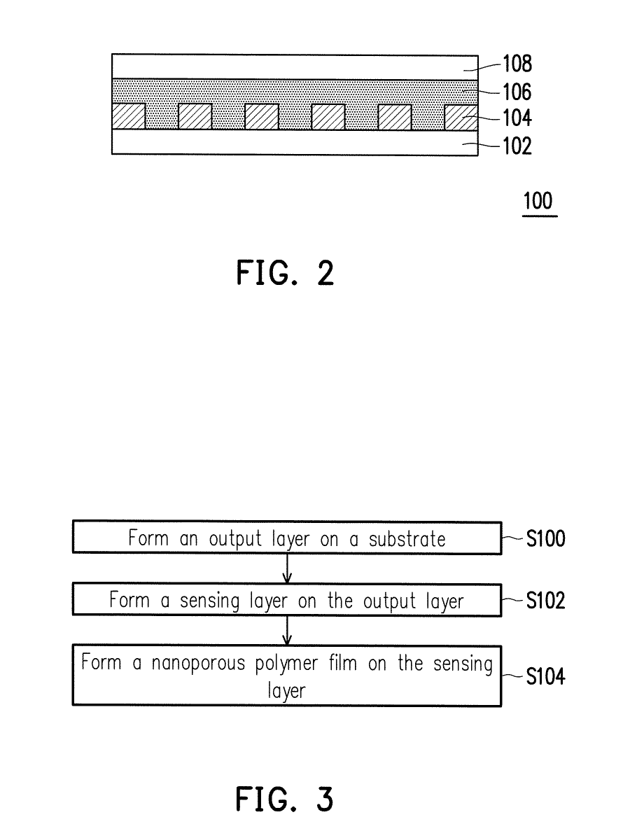

The invention does not particularly limit the constituent components and configuration of the output layer 104 provided the output layer 104 can be used to measure the physical properties (such as resistance, capacitance, or impedance) of the sensing layer 106. Such constituent components and configuration are within the scope of the invention. In some embodiments, the output layer 104 has a gap. FIG. 2 is a cross-sectional schematic diagram of a gas sensor of an embodiment of the invention. Referring to FIG. 2, the output layer 104 has a gap such that the sensing layer 106 is disposed in the gap of the output layer 104. Accordingly, the contact area between the output layer 104 and the sensing layer 106 can be greater, such that the signal strength between the output layer 104 and the sensing layer 106 can be increased to increase the sensitivity of the gas sensor. In the present embodiment, the output layer 104 is, for instance, a comb-shaped electrode. In an embodiment in which the output layer 104 is a comb-shaped electrode, the comb-shaped electrode has a body portion and a plurality of extended portions, wherein the body is extended along a direction and the extended portions are extended along another direction. Two endpoints can be selected on the comb-shaped electrode, and the resistance change of the sensing layer 106 can be obtained by measuring the physical properties (such as resistance, capacitance, or impedance) between the two endpoints of the comb-shaped electrode when the distance between the two endpoints is known so as to obtain the type, composition, or amount of the gas to be measured. In FIG. 2, the sensing layer 106 is completely filled in the gap of the output layer 104, but the invention is not limited thereto. In other embodiments, the sensing layer 106 can also be only filled in a portion of the gap of the output layer 104 and not completely filled in the gap to reduce the difficulty of the manufacturing process of the gas sensor of the invention. The output layer 104 includes a conductive material. The conductive material can be metal or metal alloy. The output layer 104 can also be a Group IV element or other types of materials. The material of the output layer 104 is, for instance, carbon powder, carbon nanotube, graphene, reduced graphene oxide, gold, platinum, silver, copper, or aluminum.

The sensing layer 106 is disposed on the output layer 104. The sensing layer 106 can sense different types of gas molecules. More specifically, the sensing layer 106 can adsorb one gas or a plurality of gases to change the resistance thereof. In other words, the sensing layer 106 is comparable to variable resistance in that the resistance thereof is changed by gas adsorption.

In some embodiments, gases sensible by the sensing layer 106 include NO₂, NH₃, H₂, CO, H₂O, ethanol, SO₂, CH₄, H₂S, O₂, NO, C₂H₂, benzene, O₃, Cl₂, methanol, acetone, or a combination thereof.

The sensing material of the sensing layer 106 can be a Group IV element or an oxide thereof, such as silicon or carbon. The carbon can be carbon nanotube or graphene. The oxide of the carbon can be graphene oxide. The sensing material of the sensing layer 106 can also be metal oxide such as zinc oxide, tin dioxide, indium oxide, tungsten trioxide, magnesium oxide, titanium dioxide, iron (II) oxide, or a combination thereof. In other embodiments, the sensing material of the sensing layer 106 can also be metal such as Au cluster. As shown in Table 1, the sensing layer 106 can sense different types of gas molecules based on different selected sensing materials.

TABLE 1

| Sensing material | Sensible analyte |
|---|---|
| Silicon | NO₂, NH₃, H₂, CO, H₂O, ethanol, O₂ |
| Carbon nanotube | NO₂, NH₃, H₂, CH₄, CO, SO₂, H₂S, O₂, NO, ethanol |
| Graphene | NO₂, NH₃, H₂, CO, H₂O, ethanol |
| Graphene oxide | NO₂, NH₃, H₂, CO, H₂O |
| Zinc oxide | NO₂, NH₃, H₂, CH₄, CO, H₂S, O₂, NO, ₂O, ethanol |
| Tin dioxide | H₂, CH₄, CO, SO₂, O₂, H₂O, ethanol, C₂H₂ |
| Indium oxide | NO₂, CH₄, CO, ethanol, C₂H₄ |
| Tungsten trioxide | NO₂, NH₃, H₂, CH₄, CO, SO₂, H₂S, O₂, NO, benzene, |
| | ethanol, O₃, Cl₂ |
| Magnesium oxide | NO₂, SO₂, O₂ |
| Titanium dioxide | NO₂, NH₃, CO, H₂O, SO₂, O₂ |
| Iron (II) oxide | Ethanol, methanol, and acetone |
| Au cluster | Volatile organic compound |

The size of the holes in the nanoporous polymer film 108 can be adjusted as needed. In other words, the size of the holes in the nanoporous polymer film 108 can decide the type of the gas to be measured that can pass through the nanoporous polymer film 108. In some embodiments, the nanoporous polymer film 108 has small holes selectively allowing smaller molecules to pass through and blocking larger molecules. For instance, smaller molecules such as water, methanol, and ethanol can pass through the holes, and debris having a larger size is excluded. By adjusting the size of the holes in the nanoporous polymer film 108, the type of the gas to be measured that can pass through the nanoporous polymer film 108 can be decided. Therefore, the nanoporous polymer film 108 can provide better protection and selectivity to the sensing layer 106 to increase the durability and performance of the gas sensor 100. The material of the nanoporous polymer film 108 is, for instance, perfluoro sulfonic acid polymer, nano cellulose, cellulose acetate, polysulfone, polyvinylamine, polyamide, polyfuran, or a combination thereof.

The diameter of the holes of the nanoporous polymer film 108 is, for instance, 0.2 nanometers to 20 nanometers. If the hole diameter of the nanoporous polymer film 108 is too large, then the selectivity of the nanoporous polymer film 108 is poor. If the hole diameter of the nanoporous polymer film 108 if too small, then the gas molecules to be measured cannot pass through the nanoporous polymer film 108 for detection. The nanoporous polymer film 108 has holes of suitable size, such that the gas sensor 100 has better performance.

In some embodiments of the invention, the thickness of the nanoporous polymer film 108 is 0.05 micrometers to 150 micrometers. If the thickness of the nanoporous polymer film 108 is too large, then the gas molecules to be measured pass through the nanoporous polymer film 108 less readily for detection. If the thickness of the nanoporous polymer film 108 is too small, then the nanoporous polymer film 108 cannot provide sufficient protection and selectivity to the gas sensor 100. The nanoporous polymer film 108 has suitable thickness, such that the gas sensor 100 has better performance.

In some embodiments, the nanoporous polymer film 108 includes an ion-based structure. The nanoporous polymer film 108 can have an ion-based functional group such that the nanoporous polymer film 108 has an ion-based structure. The nanoporous polymer film 108 having an ion-based structure carries a charge and can produce electrostatic repulsion to increase the selectivity of the nanoporous polymer film 108. In some exemplary embodiments, the ion-based functional group on the nanoporous polymer film 108 carries a positive charge, and the nanoporous polymer film 108 is a positive ion-based structure. The nanoporous polymer film 108 having a positive ion-based structure can produce electrostatic repulsion against positively-charged molecules to increase the selectivity of the nanoporous polymer film 108. In some other exemplary embodiments, the ion-based functional group on the nanoporous polymer film 108 carries a negative charge, and the nanoporous polymer film 108 is a negative ion-based structure. The nanoporous polymer film 108 having a negative ion-based structure can produce electrostatic repulsion against negatively-charged molecules to increase the selectivity of the nanoporous polymer film 108. In the present embodiment, the material of the nanoporous polymer film 108 is, for instance, perfluorosulfonic acid polymer, and the perfluorosulfonic acid polymer is, for instance, Nafion®. Nafion® has a hydrophobic skeleton and a positive ion-based end, and therefore Nafion® can form a nanoporous polymer film having a positive ion-based structure. However, the invention is not limited thereto, and the nanoporous polymer film can also be formed by other suitable materials. In some embodiments, the holes in the nanoporous polymer film 108 are formed by an ion-based structure. However, the invention is not limited thereto, and the holes in the nanoporous polymer film can also be formed by other suitable structures.

FIG. 3 is a flow chart of a method of manufacturing a gas sensor of an embodiment of the invention. Referring to FIG. 3 and FIG. 1, in step S100, the output layer 104 is formed on the substrate 102. In step S102, the sensing layer 106 is formed on the output layer 104. In step S104, the nanoporous polymer film 108 is formed on the sensing layer 106.

The gas sensor 100 can be completed by a single machine. In some embodiments, the steps of foil ling the output layer 104, forming the sensing layer 106, and forming the nanoporous polymer film 108 include 3D printing. Specifically, the step of forming the output layer 104 includes spraying the material of the output layer 104 on the substrate 102. The step of forming the sensing layer 106 includes spraying the material of the sensing layer 106 on the output layer 104. The step of forming the nanoporous polymer film 108 includes spraying the material of the nanoporous polymer film 108 on the sensing layer 106. By forming the gas sensor of the invention via 3D printing, the desired pattern can be directly printed without processes such as lithography and etching, such that tedious steps needed in known semiconductor processes can be omitted. Moreover, damage to the formed lower structures can be prevented when the upper structures are formed. Moreover, by forming the gas sensor 100 of the invention via 3D printing, only the inks needed for forming the different components are needed when each component is formed. Therefore, the issue of cross-contamination between different materials does not occur.

Different from the traditional lithography process, 3D printing provides higher degree of freedom to the configuration of the surface of the substrate 102, and can form a material on surfaces of various configurations. Therefore, the substrate 102 of the gas sensor 100 of the invention can be a flat surface or a non-planar surface. The flat surface can be a smooth surface or a rough surface. The non-planar surface can be a convex surface, a concave surface, a double concave surface, or a double convex surface. The gas sensor 100 having the non-planar substrate 102 can be disposed at a non-planar surface location or body, and therefore has a broader application scope.

In some embodiments, the step of forming the nanoporous polymer film includes 3D printing and baking. Specifically, after the material of the nanoporous polymer film is sprayed on the sensing layer, a baking step is performed. The baking step can make the structure of the nanoporous polymer film more stable to increase the durability of the gas sensor. In the present embodiment, the material of the nanoporous polymer film is, for instance, nano cellulose. However, the invention is not limited thereto, and the nanoporous polymer film can also be formed by other suitable materials.

In some embodiments, the step of forming the nanoporous polymer film can also include a solution process. The nanoporous polymer film is formed by a solution process, such that the nanoporous polymer film fit the film layer located below to provide better protection to the gas sensor. In the present embodiment, the material of the nanoporous polymer film is, for instance, perfluorosulfonic acid polymer, and the perfluorosulfonic acid polymer is, for instance, Nafion®. However, the invention is not limited thereto, and the nanoporous polymer film can also be formed by other suitable materials.

In some embodiments, the step of forming the nanoporous polymer film can include a thin film process. In the present embodiment, the material of the nanoporous polymer film is, for instance, cellulose acetate, polysulfone, polyvinylamine, polyamide, polyfuran, or a combination thereof.

Based on the above, the nanoporous polymer film of the invention has small holes selectively allowing smaller molecules to pass through and blocking larger molecules. Moreover, the nanoporous polymer film is located on the sensing layer of the gas sensor and can provide better protection to the sensing layer. Therefore, the gas sensor of the invention has very good durability and performance.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A gas sensor, comprising:
a substrate;
an output layer disposed on the substrate;
a sensing layer disposed on the output layer; and
a nanoporous polymer film disposed on the sensing layer.

2. The gas sensor of claim 1, wherein a diameter of a hole of the nanoporous polymer film is 0.2 nanometers to 20 nanometers.

3. The gas sensor of claim 1, wherein a thickness of the nanoporous polymer film is 0.05 nanometers to 150 nanometers.

4. The gas sensor of claim 1, wherein a material of the nanoporous polymer film comprises perfluoro sulfonic acid polymer, nano cellulose, cellulose acetate, polysulfone, polyvinylamine, polyamide, polyfuran or a combination thereof.

5. The gas sensor of claim 1, wherein the nanoporous polymer film comprises an ion-based structure.

6. The gas sensor of claim 1, wherein the output layer comprises an electrode.

7. The gas sensor of claim 1, wherein a surface of the substrate comprises a flat surface, a non-planar surface or a combination thereof.

8. The gas sensor of claim 1, wherein the output layer has a gap, and the sensing layer is disposed in the gap of the output layer.

9. The gas sensor of claim 1, wherein the output layer comprises a comb-shaped electrode.

10. The gas sensor of claim 1, wherein a material of the output layer comprises a conductive material, the conductive material comprises a metal or a metal alloy.

11. The gas sensor of claim 1, wherein a material of the output layer comprises carbon powder, carbon nanotube, graphene, reduced graphene oxide, gold, platinum, silver, copper or aluminum.

12. The gas sensor of claim 1, wherein a material of the sensing layer comprises a Group IV element or an oxide of the Group IV element.

13. The gas sensor of claim 1, wherein a material of the nanoporous polymer film comprises a positive ion-based structure.

14. The gas sensor of claim 1, wherein a material of the nanoporous polymer film comprises a negative ion-based structure.

15. A method of manufacturing a gas sensor, comprising:
forming an output layer on a substrate;
forming a sensing layer on the output layer; and
forming a nanoporous polymer film on the sensing layer.

16. The method of manufacturing the gas sensor of claim 15, wherein a method used in the steps of forming the output layer, forming the sensing layer, and forming the nanoporous polymer film comprises 3D printing.

17. The method of manufacturing the gas sensor of claim 15, wherein a method used in the step of forming the nanoporous polymer film comprises performing a solution process.

18. The method of manufacturing the gas sensor of claim 15, wherein a material of the nanoporous polymer film comprises perfluoro sulfonic acid polymer, nano cellulose, cellulose acetate, polysulfone, polyvinylamine, polyamide, polyfuran or a combination thereof.

19. The method of manufacturing the gas sensor of claim 15, wherein the step of forming the nanoporous polymer film on the sensing layer comprises:
forming a material for the nanoporous polymer film on the sensing layer; and
performing baking on the material to form the nanoporous polymer film.

20. The method of manufacturing the gas sensor of claim 15, wherein the step of forming the nanoporous polymer film on the sensing layer comprises a thin film process.
